(19) United States
(12) Patent Application Publication
(10) Pub. No.: US 2024/0074923 A1
(43) Pub. Date: Mar. 7, 2024

(54) DISPOSABLE DIAPER WITH ACCESSORIES

(71) Applicant: Cinthia FAJARDO, Laurelton, NY (US)

(72) Inventor: Cinthia FAJARDO, Laurelton, NY (US)

(21) Appl. No.: 17/572,787

(22) Filed: Jan. 11, 2022

(51) Int. Cl. A61F 13/84; A61F 13/495; A61F 13/505

(52) U.S. Cl. CPC A61F 13/84; A61F 13/495; A61F 13/505; A61F 2013/4955

(57) ABSTRACT

The disposable diaper with accessories is adapted for use in the capture of the eliminations and excretions of a biological entity such as an infant. The disposable diaper with accessories is worn by the biological entity. The disposable diaper with accessories comprises a master structure and a plurality of absorbent sheetings. An individual absorbent sheeting selected from the plurality of absorbent sheetings removably inserts in the master sheeting. The individual absorbent sheeting captures the eliminations and the excretions of the biological entity. The individual absorbent sheeting is a disposable structure. A first individual absorbent sheeting selected from the plurality of absorbent sheetings is replaced by a second individual absorbent sheeting selected from the plurality of absorbent sheetings after the first individual absorbent sheeting has captured the eliminations and the excretions of the biological entity.

### CROSS REFERENCES TO RELATED APPLICATIONS

Not Applicable

### STATEMENT REGARDING FEDERALLY SPONSORED RESEARCH

Not Applicable

### REFERENCE TO APPENDIX

Not Applicable

### BACKGROUND OF THE INVENTION

### Field of the Invention

The present invention relates to the field of accessories for an absorbent pad. (A61F13/84)

### SUMMARY OF INVENTION

The disposable diaper with accessories is adapted for use in the capture of the eliminations and excretions of a biological entity such as an infant. The disposable diaper with accessories is worn by the biological entity. The disposable diaper with accessories comprises a master structure and a plurality of absorbent sheetings. An individual absorbent sheeting selected from the plurality of absorbent sheetings removably inserts in the master sheeting. The individual absorbent sheeting captures the eliminations and the excretions of the biological entity. The individual absorbent sheeting is a disposable structure. A first individual absorbent sheeting selected from the plurality of absorbent sheetings is replaced by a second individual absorbent sheeting selected from the plurality of absorbent sheetings after the first individual absorbent sheeting has captured the eliminations and the excretions of the biological entity.

These together with additional objects, features and advantages of the disposable diaper with accessories will be readily apparent to those of ordinary skill in the art upon reading the following detailed description of the presently preferred, but nonetheless illustrative, embodiments when taken in conjunction with the accompanying drawings.

In this respect, before explaining the current embodiments of the disposable diaper with accessories in detail, it is to be understood that the disposable diaper with accessories is not limited in its applications to the details of construction and arrangements of the components set forth in the following description or illustration. Those skilled in the art will appreciate that the concept of this disclosure may be readily utilized as a basis for the design of other structures, methods, and systems for carrying out the several purposes of the disposable diaper with accessories.

It is therefore important that the claims be regarded as including such equivalent construction insofar as they do not depart from the spirit and scope of the disposable diaper with accessories. It is also to be understood that the phraseology and terminology employed herein are for purposes of description and should not be regarded as limiting.

### BRIEF DESCRIPTION OF DRAWINGS

The accompanying drawings, which are included to provide a further understanding of the invention are incorporated in and constitute a part of this specification, illustrate an embodiment of the invention and together with the description serve to explain the principles of the invention. They are meant to be exemplary illustrations provided to enable persons skilled in the art to practice the disclosure and are not intended to limit the scope of the appended claims.

FIG. 1 is a perspective view of an embodiment of the disclosure.

FIG. 2 is a front view of an embodiment of the disclosure.

FIG. 3 is a rear view of an embodiment of the disclosure.

FIG. 4 is a top view of an embodiment of the disclosure.

### DETAILED DESCRIPTION OF THE EMBODIMENT

The following detailed description is merely exemplary in nature and is not intended to limit the described embodiments of the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to practice the disclosure and are not intended to limit the scope of the appended claims. Furthermore, there is no intention to be bound by any expressed or implied theory presented in the preceding technical field, background, brief summary or the following detailed description.

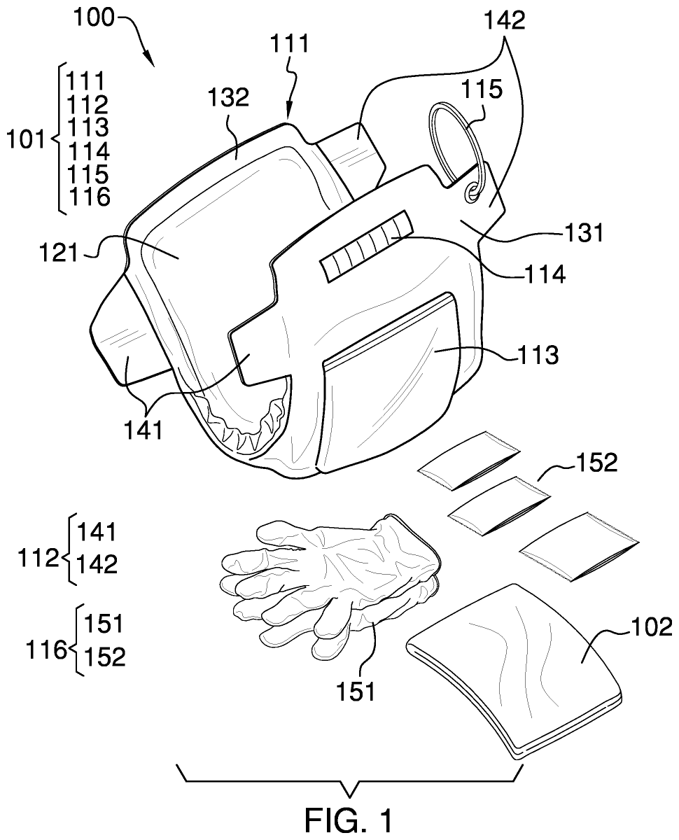

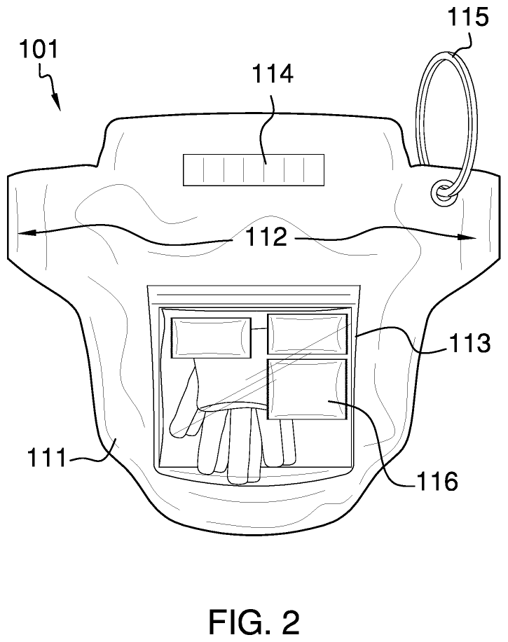

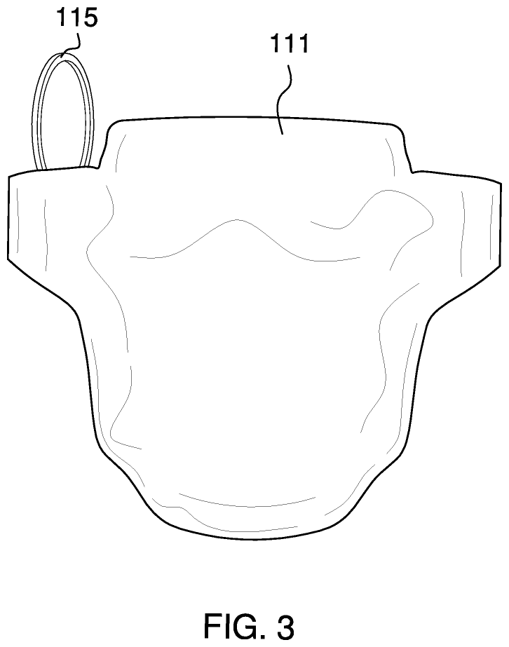

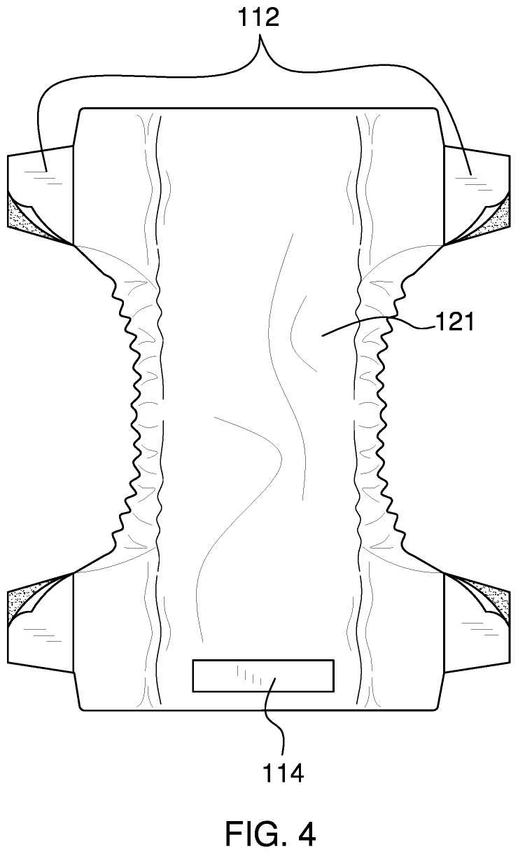

Detailed reference will now be made to one or more potential embodiments of the disclosure, which are illustrated in FIGS. 1 through 4

The disposable diaper with accessories 100 (hereinafter invention) is adapted for use in the capture of the eliminations and excretions of a biological entity such as an infant. The invention 100 is worn by the biological entity. The invention 100 comprises a master structure 101 and a plurality of absorbent sheetings 102. An individual absorbent sheeting 121 selected from the plurality of absorbent sheetings 102 removably inserts in the master structure 101. The individual absorbent sheeting 121 captures the eliminations and the excretions of the biological entity. The individual absorbent sheeting 121 is a disposable structure. A first individual absorbent sheeting 121 selected from the plurality of absorbent sheetings 102 is replaced by a second individual absorbent sheeting 121 selected from the plurality of absorbent sheetings 102 after the first individual absorbent sheeting 121 has captured the eliminations and the excretions of the biological entity.

Each of the plurality of absorbent sheetings 102 is a sheeting structure. The plurality of absorbent sheetings 102 is an absorbent structure. The plurality of absorbent sheetings 102 is a disposable structure. Each individual absorbent sheeting 121 selected from the plurality of absorbent sheetings 102 captures and stores the generated eliminations and excretions. Each individual absorbent sheeting 121 selected from the plurality of absorbent sheetings 102 is a disposable structure. Each individual absorbent sheeting 121 selected from the plurality of absorbent sheetings 102 is an interchangeable structure. The individual absorbent sheeting 121 selected from the plurality of absorbent sheetings 102 is disposed of and replaced after capturing the eliminations and excretions.

The master structure 101 is a sheeting structure. The master structure 101 is a fluid impermeable structure. An individual absorbent sheeting 121 selected from the plurality of absorbent sheetings 102 removably attaches to the master structure 101. The fluid impermeable nature of the master structure 101 contains the eliminations and excretions captured by an individual absorbent sheeting 121 within the invention 100 until the individual absorbent sheeting 121 is replaced. The master structure 101 comprises a master sheeting 111, a plurality of fastening devices 112, a pocket 113, a thermometer 114, an anchor ring 115, and a plurality of accessories 116.

The master sheeting 111 forms the sheeting structure of the master structure 101. The plurality of fastening devices 112, the pocket 113, the thermometer 114, and the anchor ring 115 attach to the master sheeting 111. Each individual absorbent sheeting 121 selected from the plurality of absorbent sheetings 102 removably attaches to the master sheeting 111. The master sheeting 111 is formed from a fluid impermeable material. The master sheeting 111 forms a fluid impermeable barrier that contains and stores the eliminations and excretions captured by the individual absorbent sheeting 121 within the invention 100. The master sheeting 111 further comprises an exterior surface 131 and an interior surface 132.

The exterior surface 131 is the surface of the master sheeting 111 that is distal from the biological entity that is generating the eliminations and excretions. The exterior surface 131 is the surface of the master sheeting 111 with the greatest surface area. The interior surface 132 is the surface of the master sheeting 111 that is proximal to the biological entity that is generating the eliminations and excretions. The interior surface 132 is the surface of the master sheeting 111 that is distal from the exterior surface 131.

The plurality of fastening devices 112 attach to the exterior surface 131 of the master sheeting 111. The pocket 113 attaches to the exterior surface 131 of the master sheeting 111. The thermometer 114 attaches to the master sheeting 111 such that the thermometer 114 measures the temperature within the interior structure of the invention 100. The thermometer 114 attaches to the master sheeting 111 such that the thermometer 114 displays the measured temperature in a manner that is visible from the exterior of the structure formed by the invention 100. The anchor ring 115 attaches to the exterior surface 131 of the master sheeting 111. The plurality of accessories 116 are stored within the pocket 113 of the master structure 101. Each individual absorbent sheeting 121 selected from the plurality of absorbent sheetings 102 removably attaches to the interior surface 132 of the master sheeting 111.

Each of the plurality of fastening devices 112 is a mechanical structure. Each of the plurality of fastening devices 112 physically secures the invention 100 to the individual designated to wear the invention 100. The plurality of fastening devices 112 further comprises a first fastening device 141 and a second fastening device 142.

The first fastening device 141 is a fastening device. The first fastening device 141 secures the invention 100 to the biological entity generating the eliminations and excretions. The first fastening device 141 attaches to the exterior surface 131 of the master sheeting 111. The second fastening device 142 is a fastening device. The second fastening device 142 secures the invention 100 to the biological entity generating the eliminations and excretions. The second fastening device 142 attaches to the exterior surface 131 of the master sheeting 111. In the first potential embodiment of the disclosure, the first fastening device 141 and the second fastening device 142 are hook and loop fasteners.

The pocket 113 is a storage space. The pocket 113 is defined elsewhere in this disclosure. The pocket 113 forms a contained space used to store the plurality of accessories 116.

The thermometer 114 is a temperature sensor. The thermometer 114 measures the temperature in the interior space of the invention 100. The thermometer 114 displays the measured temperature such that the measured temperature is visible from the exterior of the invention 100.

The anchor ring 115 is a ring structure. The anchor ring 115 attaches to the exterior surface 131 of the master sheeting 111. The anchor ring 115 forms an anchor point that allows the invention 100 to be attached to an externally provided object such as a luggage item.

Each of the plurality of accessories 116 is an accessory associated with the replacement and disposal of an individual absorbent sheeting 121 that has previously captured eliminations and excretions. Each of the plurality of accessories 116 is stored in the pocket 113 in anticipation of use. The plurality of accessories 116 further comprises a plurality of disposable gloves 151 and a plurality of pre-moistened wipes 152.

Each of the plurality of disposable gloves 151 is a glove. Each of the plurality of disposable gloves 151 forms a fluid impermeable barrier that prevents contact transfer between the eliminations and excretions and an individual replacing the individual absorbent sheeting 121 that contains the eliminations and excretions. Each of the plurality of pre-moistened wipes 152 is a pre-moistened wipe. The pre-moistened wipe is defined elsewhere in this disclosure. Each of the plurality of pre-moistened wipes 152 is moistened with a cosmetic media used for sanitation purposes.

The following definitions were used in this disclosure:

Absorbent: As used in this disclosure, absorbent is an adjective that refers to a material that is able to soak up a liquid such as water.

Accessory: As used in this disclosure, an accessory is a first object or first device that is configured for use with a second object or second device. The use of the accessory enhances the appearance or functionality of the second object or second device.

Align: As used in this disclosure, align refers to an arrangement of objects that are: 1) arranged in a straight plane or line; 2) arranged to give a directional sense of a plurality of parallel planes or lines; or, 3) a first line or curve is congruent to and overlaid on a second line or curve.

Anchor: As used in this disclosure, anchor means to hold an object firmly or securely.

Anchor Point: As used in this disclosure, an anchor point is a location to which a first object can be securely attached to a second object.

Cant: As used in this disclosure, a cant is an angular deviation from one or more reference lines (or planes) such as a vertical line (or plane) or a horizontal line (or plane).

Center: As used in this disclosure, a center is a point that is: 1) the point within a circle that is equidistant from all the points of the circumference; 2) the point within a regular polygon that is equidistant from all the vertices of the regular polygon; 3) the point on a line that is equidistant from the ends of the line; 4) the point, pivot, or axis around which something revolves; or, 5) the centroid or first moment of an area or structure. In cases where the appropriate definition or definitions are not obvious, the fifth option should be used in interpreting the specification.

Center Axis: As used in this disclosure, the center axis is the axis of a cylinder or a prism. The center axis of a prism is the line that joins the center point of the first congruent face of the prism to the center point of the second corresponding congruent face of the prism. The center axis of a pyramid refers to a line formed through the apex of the pyramid that is perpendicular to the base of the pyramid. When the center axes of two cylinder, prism or pyramidal structures share the same line they are said to be aligned. When the center axes of two cylinder, prism or pyramidal structures do not share the same line they are said to be offset.

Composite Prism: As used in this disclosure, a composite prism refers to a structure that is formed from a plurality of structures selected from the group consisting of a prism structure and a pyramid structure. The plurality of selected structures may or may not be truncated. The plurality of prism structures are joined together such that the center axes of each of the plurality of structures are aligned. The congruent ends of any two structures selected from the group consisting of a prism structure and a pyramid structure need not be geometrically similar.

Composite Textile: As used in this disclosure, a composite textile is a multilayer fabric made of two or more joined layers of textile or sheeting materials.

Congruent: As used in this disclosure, congruent is a term that compares a first object to a second object. Specifically, two objects are said to be congruent when: 1) they are geometrically similar; and, 2) the first object can superimpose over the second object such that the first object aligns, within manufacturing tolerances, with the second object.

Correspond: As used in this disclosure, the term correspond is used as a comparison between two or more objects wherein one or more properties shared by the two or more objects match, agree, or align within acceptable manufacturing tolerances.

Cosmetic Media: As used in this disclosure, a cosmetic media refers to a chemical substance that is topically applied to a biological organism. The purposes for a cosmetic media include, but are not limited to: a) cleaning the skin and the hair of the biological organism; b) changing the visual, olfactory, and tactile stimuli presented by the biological organism to other nearby biological organisms; and, c) the topical application of a pharmacologically active media.

Disk: As used in this disclosure, a disk is a prism-shaped object that is flat in appearance. The disk is formed from two congruent ends that are attached by a lateral face. The sum of the surface areas of two congruent ends of the prism-shaped object that forms the disk is greater than the surface area of the lateral face of the prism-shaped object that forms the disk. In this disclosure, the congruent ends of the prism-shaped structure that forms the disk are referred to as the faces of the disk.

Disposable: As used in this disclosure, disposable is an adjective that refers to an object that is designed and intended for a single use. Within this context, an object would be considered disposable if it is not reusable after its initial use.

Elimination: As used in this disclosure, an elimination refers to a solid phase discharge from a biological entity.

Excretion: As used in this disclosure, an excretion refers to a liquid phase discharge from a biological entity.

Exterior: As used in this disclosure, the exterior is used as a relational term that implies that an object is not contained within the boundary of a structure or a space.

Flow: As used in this disclosure, a flow refers to the passage of a fluid past a fixed point. This definition considers bulk solid materials as capable of flow.

Fluid: As used in this disclosure, a fluid refers to a state of matter wherein the matter is capable of flow and takes the shape of a container it is placed within. The term fluid commonly refers to a liquid or a gas.

Form Factor: As used in this disclosure, the term form factor refers to the size and shape of an object.

Gas: As used in this disclosure, a gas refers to a state (phase) of matter that is fluid and that fills the volume of the structure that contains it. Stated differently, the volume of a gas always equals the volume of its container.

Geometrically Similar: As used in this disclosure, geometrically similar is a term that compares a first object to a second object wherein: 1) the sides of the first object have a one to one correspondence to the sides of the second object; 2) wherein the ratio of the length of each pair of corresponding sides are equal; 3) the angles formed by the first object have a one to one correspondence to the angles of the second object; and, 4) wherein the corresponding angles are equal. The term geometrically identical refers to a situation where the ratio of the length of each pair of corresponding sides equals 1.

Hook and Loop Fastener: As used in this disclosure, a hook and loop fastener is a fastener that comprises a hook surface and a loop surface. The hook surface comprises a plurality of minute hooks. The loop surface comprises a surface of uncut pile that acts like a plurality of loops. When the hook surface is applied to the loop surface, the plurality of minute hooks fastens to the plurality of loops securely fastening the hook surface to the loop surface. A note on usage: when fastening two objects the hook surface of a hook and loop fastener will be placed on the first object and the matching loop surface of a hook and loop fastener will be placed on the second object without significant regard to which object of the two objects is the first object and which of the two objects is the second object. When the hook surface of a hook and loop fastener or the loop surface of a hook and loop fastener is attached to an object this will simply be referred to as the “hook/loop surface” with the understanding that when the two objects are fastened together one of the two objects will have a hook surface and the remaining object will have the loop surface.

Infant: As used in this disclosure, an infant refers to a human child who: 1) is under 18 months old; and, 2) has not yet learned to walk.

Interchangeable: As used in this disclosure, interchangeable refers to the ability to remove and replace an element of a structure. For example, if a first object that is attached to a structure can be removed and replaced with a second object selected from a plurality of compatible objects than the first object is said to be replaceable with both: 1) the second object; and, 2) each of the elements of compatible objects. Term interchangeable is commonly associated with tools. Interchangeable objects are often used to change the function or the performance characteristics of a tool.

Interior: As used in this disclosure, the interior is used as a relational term that implies that an object is contained within the boundary of a structure or a space.

Liquid: As used in this disclosure, a liquid refers to a state (phase) of matter that is fluid and that maintains, for a given pressure, a fixed volume that is independent of the volume of the container.

Loop: As used in this disclosure, a loop is the length of a first structure including, that is folded or curved to form a closed or nearly closed space such that a linear structure such as a line, a cord or a hook can be inserted through the space formed within the first structure. Within this disclosure, the first structure is said to be looped around the linear structure.

Negative Space: As used in this disclosure, negative space is a method of defining an object through the use of open or empty space as the definition of the object itself, or, through the use of open or empty space to describe the boundaries of an object.

One to One: When used in this disclosure, a one to one relationship means that a first element selected from a first set is in some manner connected to only one element of a second set. A one to one correspondence means that the one to one relationship exists both from the first set to the second set and from the second set to the first set. A one to one fashion means that the one to one relationship exists in only one direction.

Pan: As used in this disclosure, a pan is a hollow and prism-shaped containment structure. The pan has a single open face. The open face of the pan is often, but not always, the superior face of the pan. The open face is a surface selected from the group consisting of: a) a congruent end of the prism structure that forms the pan; and, b) a lateral face of the prism structure that forms the pan. A semi-enclosed pan refers to a pan wherein the closed end of prism structure of the pan and/or a portion of the closed lateral faces of the pan are open.

Perimeter: As used in this disclosure, a perimeter is one or more curved or straight lines that bounds an enclosed area on a plane or surface. The perimeter of a circle is commonly referred to as a circumference.

Pharmacologically Active Media: As used in this disclosure, a pharmacologically active media refers to a chemical substance that has a biochemical or physiological effect on a biological organism.

Phase: As used in this disclosure, phase refers to the state of the form of matter. The common states of matter are solid, liquid, gas, and plasma.

Pocket: As used in this disclosure, a pocket is a small pouch or storage space that is formed on or into an object. Pockets are often formed by joining a second textile or a second sheeting to a first textile or a first sheeting, respectively, by sewing or heat sealing respectively. Methods to form pockets are well-known and documented in the textile and apparel arts.

Pre-Moistened Wipe: As used in this disclosure, a pre-moistened wipe is a paper or textile is that previously moistened and that is used for cleaning purposes. By previously moistened is meant that the paper or textile is moistened before the pre-moistened wipe is packaged for storage. These previously moistened papers or textiles will remain moist until subsequent accessed. The previously moistened paper or textile can be individually wrapped for storage or can be stored in bulk. Pre-moistened wipe are commonly referred to baby wipes or wet wipes.

Prism: As used in this disclosure, a prism is a three-dimensional geometric structure wherein: 1) the form factor of two faces of the prism are congruent; and, 2) the two congruent faces are parallel to each other. The two congruent faces are also commonly referred to as the ends of the prism. The surfaces that connect the two congruent faces are called the lateral faces. In this disclosure, when further description is required a prism will be named for the geometric or descriptive name of the form factor of the two congruent faces. If the form factor of the two corresponding faces has no clearly established or well-known geometric or descriptive name, the term irregular prism will be used. The center axis of a prism is defined as a line that joins the center point of the first congruent face of the prism to the center point of the second corresponding congruent face of the prism. The center axis of a prism is otherwise analogous to the center axis of a cylinder. A prism wherein the ends are circles is commonly referred to as a cylinder.

Ring: As used in this disclosure, a ring is term that is used to describe a disk-like structure through which a negative space is formed through the faces of the disk-like structure. Rings are often considered loops.

Sacrificial Structure: As used in this disclosure, a sacrificial structure is a first object or structure that protects a second object or structure from damage. More specifically, the sacrificial structure protects the second object or structure by being damaged instead of the second object or structure.

Sheeting: As used in this disclosure, a sheeting is a material, such as a paper, textile, a plastic, or a metal foil, in the form of a thin flexible layer or layers. The sheeting forms a disk structure. The two surfaces of the sheeting with the greatest surface area are called the faces of the sheeting.

Solid: As used in this disclosure, a solid refers to a state (phase) of matter that: 1) has a fixed volume; and, 2) does not flow.

Temperature Sensor: As used in this disclosure, a temperature sensor is a device that is used to measure the temperature of an object or a space.

Topical: As used in this disclosure, topical is an adjective that is associated with a pharmacologically active media. Topical indicates that the pharmacologically active media is applied directly to the skin.

With respect to the above description, it is to be realized that the optimum dimensional relationship for the various components of the invention described above and in FIGS. 1 through 4 include variations in size, materials, shape, form, function, and manner of operation, assembly and use, are deemed readily apparent and obvious to one skilled in the art, and all equivalent relationships to those illustrated in the drawings and described in the specification are intended to be encompassed by the invention.

It shall be noted that those skilled in the art will readily recognize numerous adaptations and modifications which can be made to the various embodiments of the present invention which will result in an improved invention, yet all of which will fall within the spirit and scope of the present invention as defined in the following claims. Accordingly, the invention is to be limited only by the scope of the following claims and their equivalents.

The inventor claims:

1. A disposable diaper with accessories comprising
a master structure and a plurality of absorbent sheetings;
wherein an individual absorbent sheeting selected from the plurality of absorbent sheetings removably inserts in the master structure;
wherein the master structure comprises a master sheeting, a plurality of fastening devices, a pocket, a thermometer, an anchor ring, and a plurality of accessories;
wherein the plurality of fastening devices, the pocket, the thermometer, and the anchor ring attach to the master sheeting;
wherein each individual absorbent sheeting selected from the plurality of absorbent sheetings removably attaches to the master sheeting;
wherein the pocket contains the plurality of accessories;
wherein each of the plurality of accessories is an accessory associated with the replacement and disposal of an individual absorbent sheeting of the plurality of absorbent sheetings that has previously captured eliminations and excretions;
wherein each of the plurality of accessories is stored in the pocket in anticipation of use;
wherein the plurality of accessories further comprises a plurality of disposable gloves and a plurality of pre-moistened wipes;
wherein each of the plurality of disposable gloves is a glove;
wherein each of the plurality of disposable gloves forms a fluid impermeable barrier that prevents contact transfer between the eliminations and excretions and an individual replacing the individual absorbent sheeting that contains the eliminations and excretions;
wherein each of the plurality of pre-moistened wipes is a pre-moistened wipe;
wherein the pre-moistened wipe is defined elsewhere in this disclosure;
wherein each of the plurality of pre-moistened wipes is moistened with a cosmetic media used for sanitation purposes;
wherein the plurality of fastening devices further comprises a first fastening device and a second fastening device;
wherein the first fastening device is a fastening device;
wherein the first fastening device attaches to the exterior surface of the master sheeting;
wherein the second fastening device is a fastening device;
wherein the second fastening device attaches to the exterior surface of the master sheeting;
wherein the first fastening device and the second fastening device are hook and loop fasteners.

2. The disposable diaper with accessories according to claim 1
wherein the disposable diaper with accessories is adapted for use in capturing the eliminations and excretions of a biological entity;
wherein the individual absorbent sheeting captures the eliminations and the excretions;
wherein the individual absorbent sheeting is a disposable structure;
wherein a first individual absorbent sheeting selected from the plurality of absorbent sheetings is replaced by a second individual absorbent sheeting selected from the plurality of absorbent sheetings after the first individual absorbent sheeting has captured the eliminations and the excretions.

3. The disposable diaper with accessories according to claim 2
wherein each of the plurality of absorbent sheetings is a sheeting structure;
wherein each of the plurality of absorbent sheetings is an absorbent structure;
wherein each of the plurality of absorbent sheetings is a disposable structure.

4. The disposable diaper with accessories according to claim 3
wherein each individual absorbent sheeting selected from the plurality of absorbent sheetings captures and stores the eliminations and excretions;
wherein each individual absorbent sheeting selected from the plurality of absorbent sheetings is an interchangeable structure;
wherein the individual absorbent sheeting selected from the plurality of absorbent sheetings is disposed of and replaced after capturing the eliminations and excretions.

5. The disposable diaper with accessories according to claim 4
wherein the master structure is a fluid impermeable structure;
wherein the individual absorbent sheeting selected from the plurality of absorbent sheetings removably attaches to the master structure;
wherein a fluid impermeable nature of the master structure contains the eliminations and excretions captured by an individual absorbent sheeting within the disposable diaper with accessories until the individual absorbent sheeting is replaced.

6. (canceled)

7. The disposable diaper with accessories according to claim 5
wherein the master sheeting forms the sheeting structure of the master structure;
wherein the master sheeting is formed from a fluid impermeable material;
wherein the master sheeting forms a fluid impermeable barrier that contains and stores the eliminations and excretions captured by the individual absorbent sheeting within the disposable diaper with accessories.

8. The disposable diaper with accessories according to claim 7
wherein the master sheeting further comprises an exterior surface and an interior surface;
wherein the exterior surface of the master sheeting has a greatest surface area;
wherein the interior surface of the master sheeting that is distal from the exterior surface.

9. The disposable diaper with accessories according to claim 8
wherein the plurality of fastening devices attach to the exterior surface of the master sheeting;
wherein the pocket attaches to the exterior surface of the master sheeting;
wherein the thermometer attaches to the master sheeting such that the thermometer measures a temperature within an interior structure of the disposable diaper with accessories;
wherein the thermometer attaches to the master sheeting such that the thermometer displays the measured temperature in a manner that is visible from the exterior of the master structure formed by the disposable diaper with accessories;
wherein the anchor ring attaches to the exterior surface of the master sheeting;
wherein each individual absorbent sheeting selected from the plurality of absorbent sheetings removably attaches to the interior surface of the master sheeting.

10. The disposable diaper with accessories according to claim 9
wherein each of the plurality of fastening devices is a mechanical structure;
wherein each of the plurality of fastening devices physically secures the disposable diaper with accessories to the biological entity.

11. The disposable diaper with accessories according to claim 10
wherein the pocket is a storage space;
wherein the pocket forms a contained space used to store the plurality of accessories.

12. The disposable diaper with accessories according to claim 11
wherein the thermometer is a temperature sensor;
wherein the thermometer measures the temperature in the interior space of the disposable diaper with accessories;
wherein the thermometer displays the measured temperature such that the measured temperature is visible from the exterior of the disposable diaper with accessories.

13. The disposable diaper with accessories according to claim 12
wherein the anchor ring is a ring structure;
wherein the anchor ring attaches to the exterior surface of the master sheeting;
wherein the anchor ring forms an anchor point that allows the disposable diaper with accessories to be attached to an externally provided object.

14. (canceled)

15. (canceled)
